(19) United States
(12) Patent Application Publication
(10) Pub. No.: US 2026/0109953 A1
(43) Pub. Date: Apr. 23, 2026

(54) XENO-FREE PEHA POLYMERS FOR 3D PRINTING AND METHODS OF MAKING AND USING THE SAME

(71) Applicant: THE TRUSTEES OF INDIANA UNIVERSITY, Bloomington, IN (US)

(72) Inventors: Chien-Chi Lin, Indianapolis, IN (US); Nathan H. Dimmitt, Bloomington, IN (US); Fang-Yi Lin, Bloomington, IN (US)

(21) Appl. No.: 19/120,512

(22) PCT Filed: Apr. 25, 2023
(86) PCT No.: PCT/US23/66189

§ 371 Date: Apr. 11, 2025

Related U.S. Application Data
(60) Provisional application No. 63/415,023, filed on Oct. 11, 2022.

(51) Int. Cl. C12N 5/074

(52) U.S. Cl. CPC C12N 5/0696; C12N 2533/30; C12N 2537/10

(57) ABSTRACT

A composition comprising a hydrogel includes a polymer of formula (1), at least one repeating unit of which comprises BO₂H₂. Methods of making such hydrogels and methods of encapsulating cells using such hydrogels are also disclosed herein.

### CROSS-REFERENCE TO RELATED APPLICATIONS

This application claims priority to U.S. provisional patent application No. 63/415,023, filed on Oct. 11, 2022, and which is incorporated by reference in its entirety herein.

### STATEMENT REGARDING FEDERALLY SPONSORED RESEARCH

This invention was made with government support under CA227737 awarded by National Institutes of Health. The Government has certain rights in the invention.

### FIELD

The present disclosure provides hydrogels and methods of making and using the same.

### BACKGROUND

Hydrogels have shown promise as matrices for three-dimensional cell culture. Cells tend to adhere, spread, migrate and proliferate better in hydrogels with pronounced stress relaxation.

### SUMMARY

A need exists, therefore, for mechanisms for tuning the stress relaxation and other viscoelastic behaviors of hydrogels.

The present disclosure provides a composition comprising a hydrogel. The hydrogel may include a polymer of formula (1):

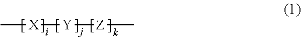

where X comprises one or more repeating units of formula (2):

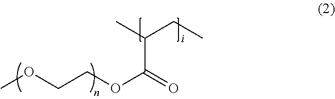

Y comprises one or more repeating units of formula (3):

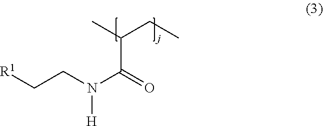

Z comprises one or more repeating units of formula (4):

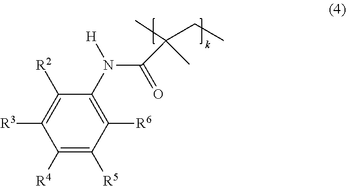

R¹ is H or OH; R²-R⁶ each independently comprise H, C, or a heteroatom; and n, i, j, and k, are each independently an integer from 1 to 4000; wherein at least one repeating unit of Z comprises BO₂H₂, and the polymer of formula (1) comprises a number average molecular weight (M_{N}) obtained via gel permeation chromatography (GPC) from 70 kDa to 250 kDa.

In embodiments, R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl. R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.

In embodiments, the other functional groups, when present, are selected from the group consisting of amino, carboxyl, hydroxy, alkoxy, ketone, aldehyde, halogen, and a combination of two or more thereof.

In embodiments, R² of at least one repeating of Z is BO₂H₂. In embodiments, R³ of at least one repeating of Z is BO₂H₂. In embodiments, Ra of at least one repeating of Z is BO₂H₂. In embodiments, R⁵ of at least one repeating of Z is BO₂H₂. In embodiments, R⁶ of at least one repeating of Z is BO₂H₂. In embodiments, R², R³, R⁴, and R⁶ of at least one repeating of Z are each H, and R⁵ of the at least one repeating of Z is BO₂H₂.

In embodiments, the composition further includes at least one pluripotent stem cell.

In embodiments, the polymer of formula (1) comprises a number average molecular weight (M_{N}) obtained via gel permeation chromatography (GPC) from 100 kDa to 250 kDa.

In embodiments, a concentration of BO₂H₂ groups present on the polymer of formula (1) is from 0.25 mmol/g to 1 mmol/g.

In embodiments, the composition further includes a diol-containing molecule. In embodiments, the diol-containing molecule comprises a polymer comprising at least one set of vicinal hydroxyl substituents. In embodiments, the polymer comprising at least one set of vicinal hydroxyl substituents comprises polyvinyl alcohol.

Another aspect of the present disclosure provides a method for culturing at least one cell on a hydrogel includes forming the composition disclosed above, adding a diol-containing molecule to the composition, adding the cell to the composition, and allowing the BO₂H₂ to react with the diol-containing molecule, thereby forming the hydrogel.

In embodiments, the cell is a stem cell. In embodiments, the cell is a pluripotent stem cell. In embodiments, the pluripotent stem cell is from a mammal. In embodiments, the mammal is a human.

In embodiments, the diol-containing molecule comprises polyvinyl alcohol.

This summary is not intended to identify key or essential features of the claimed subject matter, nor is it intended to be used as an aid in limiting the scope of the claimed subject matter. Further embodiments, forms, features, and aspects of the present disclosure shall become apparent from the description and figures provided herewith.

### BRIEF DESCRIPTION OF THE DRAWINGS

The teachings of some embodiments of the present disclosure will be better understood by reference to the description taken in conjunction with the accompanying drawings, wherein.

FIG. 1 shows schemes of RAFT polymer synthesis and hydrogel crosslinking reactions according to embodiments. (A) RAFT polymerization for synthesis of poly(OEGA-s-HEAA-s-APBA) (PEHA). (B) Reversible boronate ester bonding between poly(vinyl alcohol) (PVA) and boronic acid. “OEGA” refers to oligoethylene glycol acrylate; “HEAA” refers to N-hydroxyethyl acrylamide; and “APBA” refers to 3-aminophenylboronic acid.

FIG. 2 shows characterization data for PEHA hydrogels according to embodiments. (A) shows elastic moduli (G′) of PEHA hydrogels at different weight contents on day 0 and day 1 post-crosslinking. (B) shows loss factor (or tan(δ)=G′/G″) of PEHA hydrogels at different weight contents on day 0 and day 1 post-crosslinking.

FIG. 3 shows characterization data for PEHA hydrogels according to embodiments. (A) shows stress releaxation (G′/G′₀) of PEHA hydrogels at different weight contents. (B) shows the relaxation half-time (t_{1/2}) of PEHA hydrogels at different weight contents.

FIG. 4 shows the viability (live/dead staining) of human induced pluripotent stem cells (hiPSCs) in 3 wt % PEHA hydrogels over time.

FIG. 5 shows the immunostaining results of pluripotency genes (SSEA, OCT4) in hiPSCs maintained in 3 wt % PEHA hydrogels.

FIG. 6 shows the expression of pluripotency gene SOX2 in hiPSCs maintained in 3 wt % PEHA hydrogels.

FIG. 7 shows the shear thinning (injectable) properties of PEHA hydrogels.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless defined otherwise, all technical and scientific terms have the same meaning as is commonly understood by one of ordinary skill in the art to which this disclosure belongs. All patents, applications, published applications and other publications are incorporated by reference in their entireties. If a definition set forth in this section is contrary to, or otherwise inconsistent with, a definition set forth in a patent, application, or other publication that is incorporated by reference, the definition set forth in this section prevails over the definition incorporated by reference.

As used in the description and the appended claims, the singular forms “a,” “an,” and “the” are intended to include the plural forms as well, unless the context clearly indicates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of +20%, +10%, +5%, +1%, +0.5%, or even +0.10% of the specified amount. To provide a more concise description, some of the quantitative expressions are not qualified with the term “about.” It is understood that, whether the term “about” is used explicitly or not, every quantity is meant to refer to the actual given value, and it is also meant to refer to the approximation to such given value that would reasonably be inferred based on the ordinary skill in the art, including equivalents and approximations due to the experimental and/or measurement conditions for such given value.

The terms “including,” “containing,” and “comprising” are used in their open, non-limiting sense. The transitional phrase “consisting essentially of” means that the scope of a claim is to be interpreted to encompass the specified materials or steps recited in the claim, “and those that do not materially affect the basic and novel characteristic(s)” of the claimed subject matter. See In re Herz, 537 F.2d 549, 551-52, 190 USPQ 461, 463 (CCPA 1976) (emphasis in the original); see also MPEP § 2111.03 (9^{th} edition, 10^{th} revision).

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the metabolic biomarkers represented by the variables are specifically embraced by the present disclosure just as if each and every combination was individually and explicitly disclosed. In addition, all subcombinations of the chemical groups listed in the embodiments describing such variables are also specifically embraced by the present disclosure just as if each and every such sub-combination of metabolic biomarkers was individually and explicitly disclosed.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

As used herein, the term “mammal” refers to, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

As used herein, the term “stem cell” refers to a cell from which other types of cells develop. Stem cells are generally broadly classified into one of three groups: embryonic stem cells, adult stem cells, and induced pluripotent stem cells. Embryonic stem cells are capable of differentiating into any cell type and are thus pluripotent. Adult stem cells may differentiate into one of several cell types, but not all cell types, and are thus multipotent. Induced pluripotent stem cells are stem cells that have been created by inducing adult cells to revert to stem cells. Induced pluripotent stem cells created in this manner may then differentiate into any cell type. Thus, as used herein, the term “pluripotent stem cell” may refer to either embryonic stem cells, induced pluripotent stem cell, or both.

Nuclear magnetic resonance (NMR) spectroscopy is a technique for determining the molecular structure and concentration of molecules. In the NMR analysis of compositions described herein, the compositions or one or more components thereof are maintained in solution and then placed in a large magnetic field. The magnetic field is typically generated by a cryogenically cooled superconducting magnet, but lower magnetic fields including those generated by room temperature electromagnets or rare earth magnets can also be used. The samples are exposed to a band of radiofrequency (RF) waves which are absorbed by the molecules and excite the nuclei of the molecules. The typical focus of these experiments is on the excitation of hydrogen nuclei (also referred to as protons) and nuclei of carbon, in particular the C-13 isotope. After the RF excitation is turned off, a receiver coil then detects the RF energy that is released by the nuclei as they return from the excited state. Following Fourier transformation of these signals, an NMR spectrum is generated containing spectral peaks that, in different combinations, represent the identity of the molecules in solution. The resulting NMR spectrum can then be compared with reference spectra to both identify and quantify the molecules in the solution.

Gel permeation chromatography (GPC), sometimes referred to as “gas permeation chromatography” interchangeably, is an analytical method to characterize molecular weights (MW) and dispersity index (D) of polymers. GPC is based on size-exclusion chromatography, where the polymers are loaded on the column and separated based on their hydrodynamic volume. Principly, porous beads are packed in the GPC column and the polymers are eluted by organic solvent through the column. Smaller polymers can enter the pores of the beads more easily than larger polymers, hence increasing their retention time in the column. As such, larger polymers are eluted out first while smaller polymers are retained in the column for a longer time. Based on the retention time and elution profiles, polymer molecular weights and dispersity can be calculated.

In an aspect of the disclosure, there is provided a composition comprising a hydrogel. The hydrogel includes a polymer of formula (1):

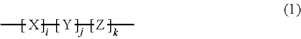

where X comprises one or more repeating units of formula (2):

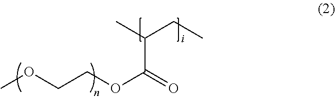

Y comprises one or more repeating units of formula (3):

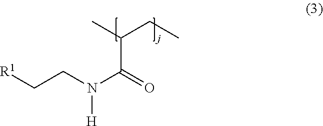

Z comprises one or more repeating units of formula (4):

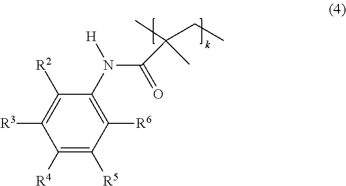

R¹ is H or OH; R²-R⁶ each independently comprise H, C, or a heteroatom; and n, i, j, and k, are each independently an integer from 2 to 4000; wherein at least one repeating unit of Z comprises BO₂H₂, and the polymer of formula (1) comprises a number average molecular weight (M_{N}) obtained via gel permeation chromatography (GPC) from 70 kDa to 250 kDa.

In embodiments, the polymer of formula (1) may be a random copolymer. In other embodiments, the polymer of formula (1) may be a block copolymer. As a random copolymer, X, Y, and Z, may be bonded to one another in any order. That is, in one portion of the polymer of formula (1), there may be one or more repeat units of X, followed by one or more repeat units of Y, followed by one or more repeat units of Z. In other portions of the polymer of formula (1), there may be one or more repeat units of Y, followed by one or more repeat units of X, followed by one or more repeat units of Z. In other portions of the polymer of formula (1), there may be one or more repeat units of Y, followed by one or more repeat units of X, followed by one or more repeat units of Y, followed by one or more repeat units of Z. In other portions of the polymer of formula (1), there may be one or more repeat units of X, followed by one or more repeat units of Y, followed by one or more repeat units of X, followed by one or more repeat units of Z. All other permutations of the binding order of X, Y, and Z are envisioned.

Additional components may be present in the composition. For instance, a diol may be added in certain embodiments. In embodiments, the diol may be a 1,2-diol. Exemplary 1,2-diols include, but are not limited to, 1,2-ethanediol, propane-1,2-diol, and dopamine. Furthermore, in some embodiments, the diol functionality may be installed on the polymer of formula (1). One convenient location for installing the diol functionality is at the terminal hydroxyl of Y of formula (1). That is, when R¹ is OH, the OH may be reacted with an appropriate diol, such as dopamine, either before or after the polymerization forming the polymer of formula (1). In some embodiments, the diol-containing molecule comprises a polymer comprising at least one set of vicinal hydroxyl substituents. In some embodiments, the polymer comprising at least one set of vicinal hydroxyl substituents comprises polyvinyl alcohol. Such diols may be useful in gel formation through a crosslinking reaction using boronate-diol bond formation.

In embodiments, R²-R⁶ each independently comprise H, C, or a heteroatom. However, at least one of R²-R⁶ is BO₂H₂. In embodiments, R²-R⁶ may each independently be H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl. In embodiments, R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups. In embodiments, the other functional groups may include amino, carboxyl, hydroxy, alkoxy, ketone, aldehyde, halogen, and a combination of two or more thereof.

In embodiments, R² is BO₂H₂. In embodiments, R³ is BO₂H₂. In embodiments, R⁴ is BO₂H₂. In embodiments, R⁵ is BO₂H₂. In embodiments, R⁶ is BO₂H₂. In embodiments, R², R³, R⁴, and R⁶ are each H, and R⁵ is BO₂H₂. Without intending to be bound by any particular theory, it is believed that the BO₂H₂ groups may facilitate dynamic covalent bonding via formation of boronate ester bonds with diols.

In embodiments, the concentration of the BO₂H₂ groups present on the polymer of formula (1) is from 0.25 nmol/g to 1 mmol/g. That is, the concentration of the BO₂H₂ groups present on the polymer of formula (1) is from 0.25 mmol/g to 0.95 mmol/g, from 0.25 mmol/g to 0.90 mmol/g, from 0.25 mmol/g to 0.85 mmol/g, from 0.25 mmol/g to 0.8 mmol/g, from 0.25 mmol/g to 0.75 mmol/g, from 0.25 mmol/g to 0.7 mmol/g, from 0.25 mmol/g to 0.65 mmol/g, from 0.25 mmol/g to 0.6 mmol/g, from 0.25 mmol/g to 0.55 mmol/g, from 0.25 mmol/g to 0.5 mmol/g, from 0.25 mmol/g to 0.45 mmol/g, from 0.25 mmol/g to 0.4 mmol/g, from 0.25 mmol/g to 0.35 mmol/g, from 0.25 mmol/g to 0.3 mmol/g, from 0.3 mmol/g to 0.95 mmol/g, from 0.35 mmol/g to 0.95 mmol/g, from 0.4 mmol/g to 0.95 mmol/g, from 0.45 mmol/g to 0.95 mmol/g, from 0.5 mmol/g to 0.95 mmol/g, from 0.55 mmol/g to 0.95 mmol/g, from 0.6 mmol/g to 0.95 mmol/g, from 0.65 mmol/g to 0.95 mmol/g, from 0.7 mmol/g to 0.95 mmol/g, from 0.75 mmol/g to 0.95 mmol/g, from 0.8 mmol/g to 0.95 mmol/g, from 0.85 mmol/g to 0.95 mmol/g, or even from 0.9 mmol/g to 0.95 mmol/g. Without intending to be bound by any particular theory, it is believed that a lower concentration of BO₂H₂ groups may lead to a low degree of stress-relaxation. Further, it is believed that a higher concentration of BO₂H₂ groups may reduce the solubility of the polymer.

In embodiments, n, i, j, and k, are each independently an integer from 1 to 5000. For example, n, i, j, and k, may each independently be an integer from 1 to 4500, 1 to 4000, 2 to 4000, 1 to 3500, from 1 to 3000, from 1 to 2500, from 1 to 2000, from 1 to 1500, from 1 to 1000, from 1 to 500, from 1 to 450, from 1 to 400, from 1 to 350, from 1 to 300, from 1 to 250, from 1 to 200, from 1 to 150, from 1 to 100, from 1 to 50, from 50 to 5000, from 100 to 5000, from 150 to 5000, from 200 to 5000, from 250 to 5000, from 300 to 5000, from 350 to 5000, from 400 to 5000, from 450 to 5000, from 500 to 5000, from 1000 to 5000, from 1500 to 5000, from 2000 to 5000, from 2500 to 5000, from 3000 to 5000, from 3500 to 5000, from 4000 to 5000, or even from 4500 to 5000.

In embodiments, the polymer of formula (1) may have a number average molecular weight (M_{N}), obtained via GPC, from 70 kDa to 250 kDa. That is, the M_{N} may be from 75 kDa to 250 kDa, from 80 kDa to 250 kDa, from 85 kDa to 250 kDa, from 90 kDa to 250 kDa, from 95 kDa to 250 kDa, from 100 kDa to 250 kDa, from 105 kDa to 250 kDa, from 110 kDa to 250 kDa, from 115 kDa to 250 kDa, from 120 kDa to 250 kDa, from 125 kDa to 250 kDa, from 130 kDa to 250 kDa, from 135 kDa to 250 kDa, from 140 kDa to 250 kDa, from 145 kDa to 250 kDa, from 150 kDa to 250 kDa, from 155 kDa to 250 kDa, from 160 kDa to 250 kDa, from 165 kDa to 250 kDa, from 170 kDa to 250 kDa, from 175 kDa to 250 kDa, from 180 kDa to 250 kDa, from 185 kDa to 250 kDa, from 190 kDa to 250 kDa, from 195 kDa to 250 kDa, from 200 kDa to 250 kDa, from 205 kDa to 250 kDa, from 210 kDa to 250 kDa, from 215 kDa to 250 kDa, from 220 kDa to 250 kDa, from 225 kDa to 250 kDa, from 230 kDa to 250 kDa, from 235 kDa to 250 kDa, from 240 kDa to 250 kDa, from 245 kDa to 250 kDa, from 70 kDa to 245 kDa, from 70 kDa to 240 kDa, from 70 kDa to 235 kDa, from 70 kDa to 230 kDa, from 70 kDa to 225 kDa, from 70 kDa to 220 kDa, from 70 kDa to 215 kDa, from 70 kDa to 210 kDa, from 70 kDa to 205 kDa, from 70 kDa to 200 kDa, from 70 kDa to 195 kDa, from 70 kDa to 190 kDa, from 70 kDa to 185 kDa, from 70 kDa to 180 kDa, from 70 kDa to 175 kDa, from 70 kDa to 170 kDa, from 70 kDa to 165 kDa, from 70 kDa to 160 kDa, from 70 kDa to 155 kDa, from 70 kDa to 150 kDa, from 70 kDa to 145 kDa, from 70 kDa to 140 kDa, from 70 kDa to 135 kDa, from 70 kDa to 130 kDa, from 70 kDa to 125 kDa, from 70 kDa to 120 kDa, from 70 kDa to 115 kDa, from 70 kDa to 110 kDa, from 70 kDa to 105 kDa, from 70 kDa to 100 kDa, from 70 kDa to 95 kDa, from 70 kDa to 90 kDa, from 70 kDa to 85 kDa, from 70 kDa to 80 kDa, or even from 70 kDa to 75 kDa. Without intending to be bound by any particular theory, it is believed that hydrogel crosslinking may be inefficient if the M_{N} is too low. Further, it is believed that too high of a M_{N} may limit the useability of hydrogels in vivo.

In another aspect of the disclosure, there is provided a method for making a composition comprising a hydrogel. The method includes forming a polymer of formula (1) via a radical process and forming a crosslinked hydrogel using boronate-diol bond formation.

In embodiments, the radical process is reversible addition-fragmentation chain transfer (“RAFT”). In embodiments, an initiator is used to initiate the RAFT polymerization. Initiatiors are known. In embodiments, the initiator may be an azo series catalyst including, but not limited to, 4′-Azobis-4-cyanovaleric acid (ACVA), 2′-Azobis-isobutyronitrile (AIBN), Azobis-2,4-dimethylvaleronitrile (ADVN), and 2′-Azobis-2-methylbutyronitrile (AMBN). In addition to the initiator, the RAFT polymerization may also use a RAFT agent. Commonly used RAFT agents include thiocarbonylthio compounds such as dithioesters, dithiocarbamates, trithiocarbonates, and xanthates. A literature review regarding RAFT polymerization is available in Perrier, “50^{th} Anniversary Perspective: RAFT Polymerization-A User Guide,” Macromolecules, vol. 50, pp. 7433-47 (2017), the entire content of which is incorporated herein by reference.

In embodiments, the method for making a composition comprising a hydrogel includes adding at least one cell to the composition. In embodiments, the cell is a pluripotent stem cell.

In embodiments, the pluripotent stem cell is from a mammal. In embodiments, the pluripotent stem cell is from a human.

In embodiments, the composition comprising the hydrogel may have an elastic modulus (G′) from 2 kPa to 10 kPa. That is, G′ may be from 2 kPa to 9 kPa, from 2 kPa to 8 kPa, from 2 kPa to 7 kPa, from 2 kPa to 6 kPa, from 2 kPa to 5 kPa, from 2 kPa to 4 kPa, from 2 kPa to 3 kPa, from 3 kPa to 10 kPa, from 4 kPa to 10 kPa, from 5 kPa to 10 kPa, from 6 kPa to 10 kPa, from 7 kPa to 10 kPa, from 8 kPa to 10 kPa, from 9 kPa to 10 kPa. Without intending to be bound by any particular theory, it is believed that an undesirably weak hydrogel may be obtained when G′ is too low. Further, it is believed that may be too stiff if G′ is too high. Hydrogels that are either too weak or too stiff are not physiologically relevant.

In embodiments, the composition comprising the hydrogel may have a tan 6 from 0.01 to 0.15. That is, the tan 6 may be be from 0.01 to 0.14, from 0.01 to 0.13, from 0.01 to 0.12, from 0.01 to 0.11, from 0.01 to 0.1, from 0.01 to 0.09, from 0.01 to 0.08, from 0.01 to 0.07, from 0.01 to 0.06, from 0.01 to 0.05, from 0.01 to 0.04, from 0.01 to 0.03, from 0.01 to 0.02, from 0.02 to 0.15, from 0.03 to 0.15, from 0.04 to 0.15, from 0.05 to 0.15, from 0.06 to 0.15, from 0.07 to 0.1, from 0.08 to 0.15, from 0.09 to 0.15, from 0.1 to 0.15, from 0.11 to 0.15, from 0.12 to 0.15, from 0.13 to 0.15, or even from 0.14 to 0.15. Without intending to be bound by any particular theory, tan 6 is a measurement of hydrogel viscoelasticity. Similar to G′, there is an optimal range for soft tissues. Materials with too low or too high tan 6 do not mimic tissues in the body, and are therefore not physiologically relevant.

In embodiments, the composition comprising the hydrogel may have a stress-relaxation halftime (t_{1/2}) from 10 seconds to 100 seconds. That is, the t_{1/2} may be from 10 seconds to 90 seconds, from 10 seconds to 85 seconds, from 10 seconds to 80 seconds, from 10 seconds to 75 seconds, from 10 seconds to 70 seconds, from 10 seconds to 65 seconds, from 10 seconds to 60 seconds, from 10 seconds to 55 seconds, from 10 seconds to 50 seconds, from 10 seconds to 45 seconds, from 10 seconds to 40 seconds, from 10 seconds to 35 seconds, from 10 seconds to 30 seconds, from 10 seconds to 25 seconds, from 10 seconds to 20 seconds, from 10 seconds to 15 seconds, from 20 seconds to 100 seconds, from 25 seconds to 100 seconds, from 30 seconds to 100 seconds, from 35 seconds to 100 seconds, from 40 seconds to 100 seconds, from 45 seconds to 100 seconds, from 50 seconds to 100 seconds, from 55 seconds to 100 seconds, from 60 seconds to 100 seconds, from 65 seconds to 100 seconds, from 70 seconds to 100 seconds, from 75 seconds to 100 seconds, from 80 seconds to 100 seconds, from 85 seconds to 100 seconds, from 90 seconds to 100 seconds, or even from 95 seconds to 100 seconds. Without intending to be bound by any particular theory, tv 2 is a measurement of hydrogel viscoelasticity. Similar to G′ and tan 6, there is an optimal range for soft tissues. Materials with too low or too high t_{1/2} do not mimic tissues in the body, and are therefore not physiologically relevant.

In another aspect of the disclosure, there is provided a method for culturing at least one cell on a hydrogel. The method includes forming any embodiment of the composition disclosed above and then adding a diol-containing molecule to the composition. Further, the cell is added to the composition. Notably, the diol-containing compound and cell may be added to the composition concurrently or sequentially. That is, the diol-containing compound and cell may be added at the same time, the diol-containing compound may be added first, or the cell may be added first. Once the diol-containing compound and cell are both added to the composition, the BO₂H₂ of the polymer of formula (1) can then be allowed to react with the diol-containing molecule, thereby forming the hydrogel.

In some embodiments, the polymer composition and the diol-containing molecule are mixed together in various proportions ranging from 100:1 to 1:100. That is, the polymer composition and the diol-containing molecule may be mixed together in a proportion including 100:1, 95:1, 90:1, 85:1, 80:1, 75:1, 70:1, 65:1 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, up to 1:100. The proportions of each component may be determined based upon the stoichiometric proportions of reactive groups necessary to optimize the reactivity of the reactants, and other practical considerations that are well within the purview of a person of ordinary skill in the art.

In some embodiments, the diol-containing molecule comprises a polymer comprising at least one set of vicinal hydroxyl substituents. In some embodiments, the polymer comprising at least one set of vicinal hydroxyl substituents comprises polyvinyl alcohol. Such diols may be useful in gel formation through a crosslinking reaction using boronate-diol bond formation.

In addition to the aspects and embodiments described and provided elsewhere in the present disclosure, the following non-limiting list of embodiments are also contemplated.
1. A composition comprising a hydrogel, the hydrogel comprising a polymer of formula (1):

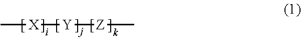

where
   X comprises one or more repeating units of formula (2):

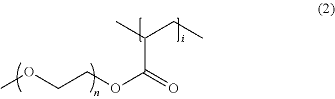

Y comprises one or more repeating units of formula (3):

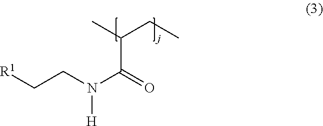

Z comprises one or more repeating units of formula (4):

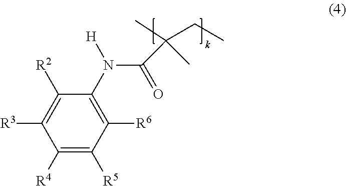

R¹ is H or OH;
R²-R⁶ each independently comprise H, C, or a heteroatom; and
n, i, j, and k, are each independently an integer from 1 to 4000;
wherein
   at least one repeating unit of Z comprises BO₂H₂; and
   the polymer of formula (1) comprises a number average molecular weight (M_{N}) obtained via gel permeation chromatography (GPC) from 70 kDa to 250 kDa.
2. The composition of clause 1, wherein R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl;
   where R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.
3. The composition of clause 2, wherein the other functional groups, when present, are selected from the group consisting of amino, carboxyl, hydroxy, alkoxy, ketone, aldehyde, halogen, and a combination of two or more thereof.
4. The composition of any one of clauses 1 to 3, wherein R² of at least one repeating of Z is BO₂H₂.
5. The composition of any one of clauses 1 to 4, wherein R³ of at least one repeating of Z is BO₂H₂.
6. The composition of any one of clauses 1 to 5, wherein R⁴ of at least one repeating of Z is BO₂H₂.
7. The composition of any one of clauses 1 to 6, wherein R⁵ of at least one repeating of Z is BO₂H₂.
8. The composition of any one of clauses 1 to 7, wherein R⁶ of at least one repeating of Z is BO₂H₂.
9. The composition of clause 7, wherein R², R³, R⁴, and R⁶ of at least one repeating of Z are each H, and R⁵ of the at least one repeating of Z is BO₂H₂.
10. The composition of any one of clauses 1 to 9, further comprising at least one pluripotent stem cell.
11. The composition of any one of clauses 1 to 10, wherein the polymer of formula (1) comprises a number average molecular weight (M_{N}) obtained via gel permeation chromatography (GPC) from 100 kDa to 250 kDa.
12. The composition of any one of clauses 1 to 11, wherein a concentration of BO₂H₂ groups present on the polymer of formula (1) is from 0.25 mmol/g to 1 mmol/g.
13. The composition of any one of clauses 1 to 12, further comprising a diol-containing molecule.
14. The composition of clause 13, wherein the diol-containing molecule comprises a polymer comprising at least one set of vicinal hydroxyl substituents.
15. The composition of clause 14, wherein the polymer comprising at least one set of vicinal hydroxyl substituents comprises polyvinyl alcohol.
16. A method for culturing at least one cell on a hydrogel, the method comprising:
   forming the composition of any one of clauses 1 to 15;
   adding a diol-containing molecule to the composition;
   adding the cell to the composition; and
   allowing the BO₂H₂ to react with the diol-containing molecule, thereby forming the hydrogel.
17. The method of clause 16, wherein the cell is a stem cell.
18. The method of clause 16 or clause 17, wherein the cell is a pluripotent stem cell.
19. The method of clause 18, wherein the pluripotent stem cell is from a mammal.
20. The method of clause 19, wherein the mammal is a human.
21. The method of any one of clauses 16-20, wherein the diol-containing molecule comprises polyvinyl alcohol.

### EXAMPLES

Examples related to the present disclosure are described below. In most cases, alternative techniques can be used. The examples are intended to be illustrative and are not limiting or restrictive of the scope of the invention as set forth in the claims.

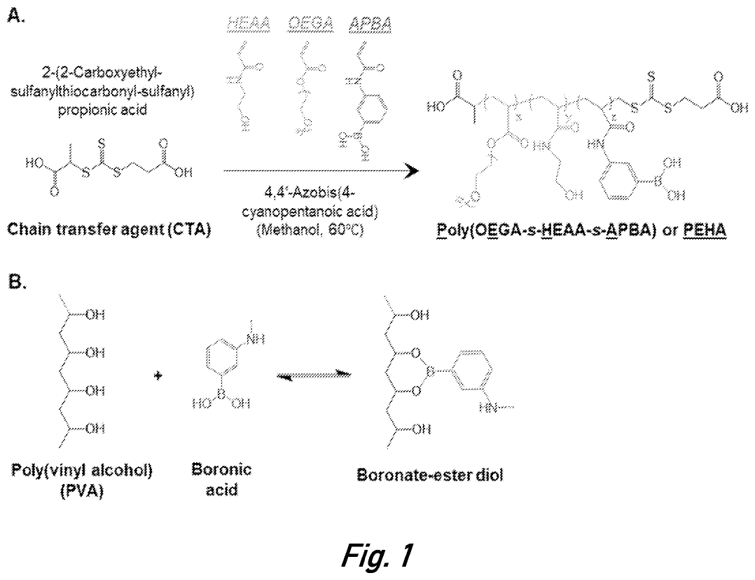

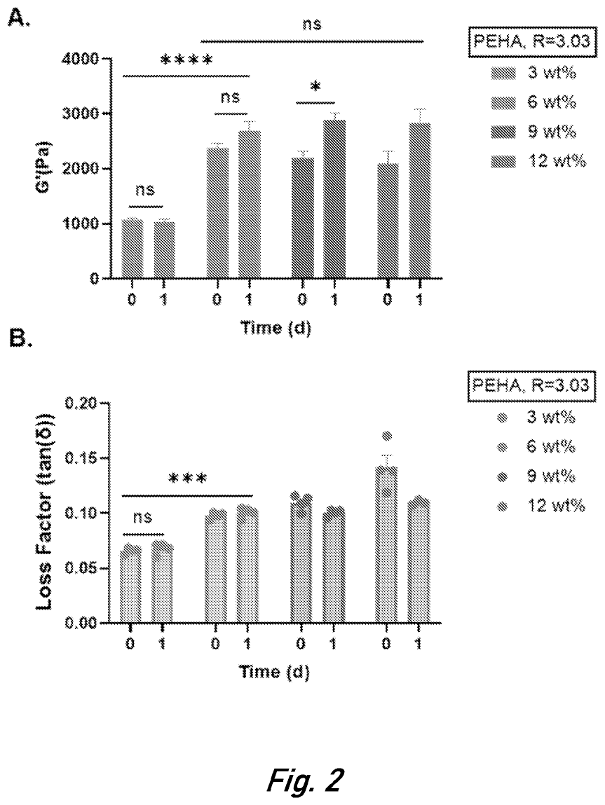

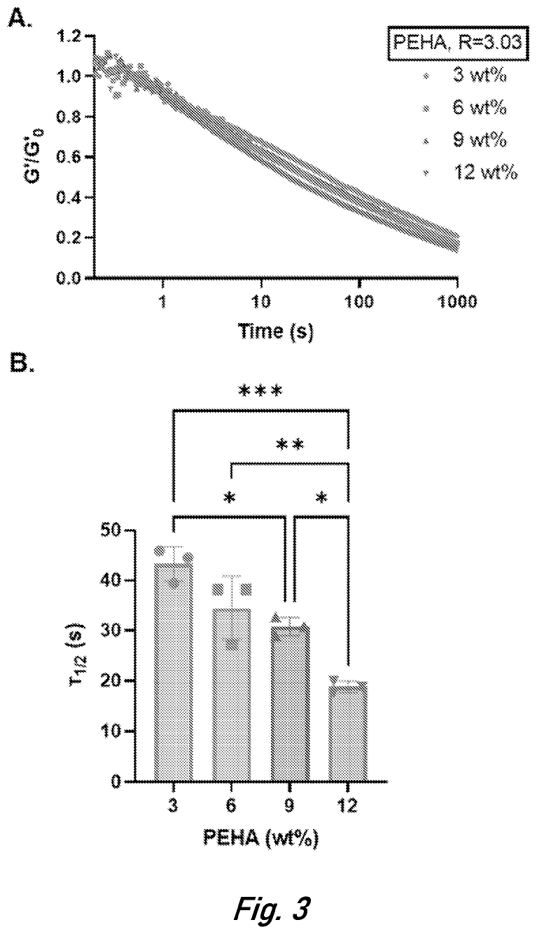

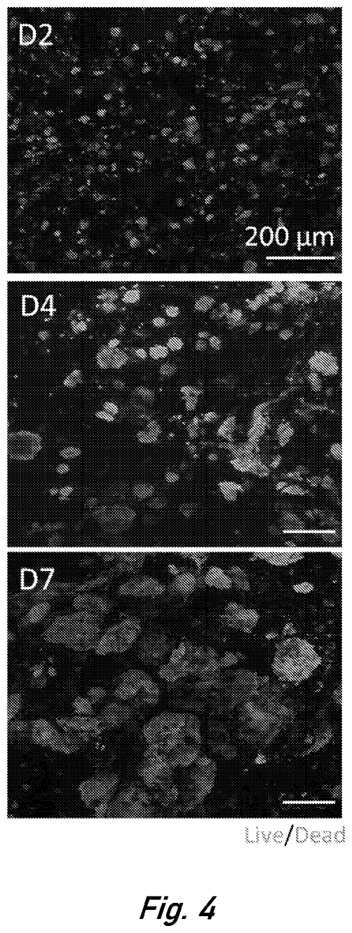

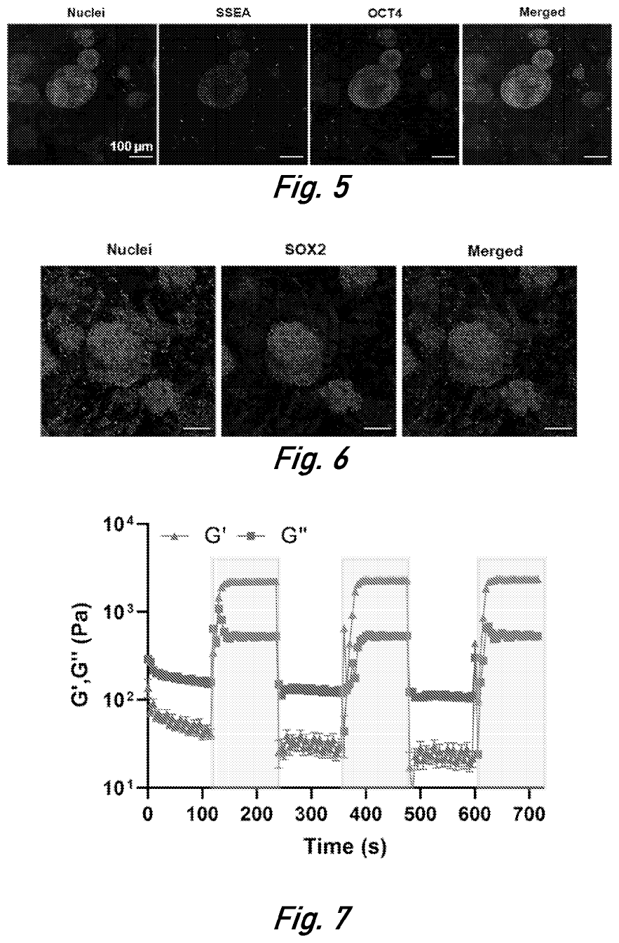

To study the design of new compositions comprising a polymer of formula (1), compositions comprising a macromer poly(oligo(ethylene glycol) acrylate-s-hydroxyethyl acrylate-s-acrylamidophenylboronic acid (“poly(OEGA-s-HEAA-s-APBA” or “PEHA”), in accordance with embodiments described above, was synthesized. As shown in FIG. 1, PEHA contains pendant boronic acid groups for forming boronate-diol bonding with diol-containing molecules, such as poly(vinyl alcohol) (PVA).

### Materials and Methods

2-(2-Carboxyethylsulfanylthiocarbonylsulfanyl)propionic acid (CPA, 95%, Sigma-Aldrich), methanol (ACS reagent, 99.8%, Sigma-Aldrich), N,N′-diisopropylcarbodiimide (DIC, Chem-Impex), 3,4-dihydroxyphenylacetic acid (DOPAC, Ambeed), 4-dimethylaminopyridine (DMAP, Aldrich), dimethylformamide (DMF, anhydrous, Alfa Aesar), lithium phenyl-2,4,6-trimethylbenzoylphosphinate (LAP, ≥95%, Sigma-Aldrich), 4-arm thiolated PEG (PEG4SH, 10 kDa, Laysan Bio), and pyridine (Fisher) were used as received. 4,4′-Azobis(4-cyanovaleric acid) (ACVA, 98%, Sigma-Aldrich) was recrystallized from methanol. Oligo(ethylene glycol) methyl ether acrylate (OEGA, M_{N}˜480 g/mol, Sigma-Aldrich) and N-hydroxyethyl acrylamide (HEAA, 97%, Sigma-Aldrich) were passed through the inhibitor remover column prior to conducting the polymerization. 3-acrylamidophenylboronic acid (AAPBA) was obtained by reacting 3-aminophenylboronic acid (APBA) with acryloyl chloride using known procedures. Thiolated gelatin (GelSH) was synthesized using gelatin, ethylene diamine, and Traut's reagent. The thiol concentration of GelSH was 0.22 mmol/g as determined by Ellman's assay.

Data were presented as mean±SEM. One-way ANOVA was used to determine the statistical significance between groups when p<0.05.

### Example 1: Polymer Synthesis and Characterization

PEHA polymers were synthesized via RAFT polymerization. For an exemplary PEHA synthesis, CPA (50 mg, 194 mol), HEAA (1.3 g, 11.67 mmol, 60 equiv.), AAPBA (1.5 g, 7.78 mmol, 40 equiv.), OEGA (7 g, 14.58 mmol, 75 equiv.), ACVA (10.9 mg, 39 μmol, 0.2 equiv.), and methanol (19.7 g) were charged in a reaction vial with a stir bar. The reaction was purged under nitrogen for 15 minutes followed by reacting at 60° C. for 8 hours. The product was dialyzed in regenerated cellulose (MWCO=3.5 k) bag against pure water for 3 days and subsequently lyophilized with yield ≥90%. The feed ratio between [CTA]:[ACVA] is 5:1. The monomeric feed ratio between [OEGA]:[HEAA]:[AAPBA] is 225:180:120. Thus the overall monomeric feed ratio of PEHA [CTA]:[HEAA]:[OEGA]:[ACVA] is 1:225:180:120:0.2.

The reaction conversion and final composition was examined by ¹H NMR (Bruker, 500 MHz) in deuterated DMSO-d₆.

Prior to molecular weight characterization by gel permeation chromatography (GPC), the boronic acid of PEHA was protected by pinacol using conventional procedures to eliminate potential interaction between polymers and GPC columns. Briefly, pinacol and PEHA were combined in a reaction vessel and heated to 60° C. The reaction continued under agitation for 1 hour. The product was dialyzed using MWCO=3.5 kDa regenerated cellulose membrane in methanol for 1 day. The protected product was recovered after solvent evaporation.

The number-average molecular weight of polymers and the corresponding dispersity were characterized by Agilent 1100 high-performance liquid chromatography (HPLC) system from Cambridge Polymer Group. The system was run at 40° C. in DMF with 0.1% lithium bromide at a flow rate of 1 mL/min. The system was equipped with a UV/Vis detector and a refractive index detector, 3 Agilent PLgel Mixed-C columns and calibrated by poly(methyl methacrylate) standards. Samples were prepared at 2 mg/mL and passed through a 0.45 m PTFE filter before testing.

The concentration of boronic acid in the final product of PEHA was determined by alizarin red (ARS) using a conventional protocol. Briefly, the content of boronic acid in PEHA was determined using AAPBA monomer as the standard. AAPBA standard solutions (starting from 500 μM) and PEHA sample were prepared in phosphate buffer solution (PBS). Alizarin red S sodium salt (ARS) was prepared in PBS at 0.3 mM. In a 96-well microplate, 125 μL ARS solution was combined with equal volume of standard or sample (n=2). The solution was incubated at room temperature for 30 minutes and read between 400-800 nm at 1 nm interval. The concentration-dependent λₘₐₓ was shifted from 515 nm to 456 nm under different level of ARS and boronic acid complexation.

Discussion: PEHA polymers with high molecular weights were synthesized using only three monomers: OEGA, HEAA, and APBA. As shown in FIG. 1, PEHA was provided borinic acid for forming boronate-ester-diol bonds with 1,2-diols on PVA. The PEHA polymers were analyzed by GPC and summarized as follows: conversion of RAFT polymerization 76.56%; number-average molecular weight (M_{N}): 95.6±0.9 kDa; dispersity: 1.64±0.06; boronic acid concentration: 4 mmol/wt %.

Due to the overlap of HEAA and OEGA ester peaks at 63.0-4.3, NMR spectra only gave qualitative results. The concentration of boronic acid in PEHA was therefore quantified by its strong complexation with ARS, which induced color change from red to yellow when complexing, using conventional methods. It was determined that PEHA contains 0.54 mmol/g boronic acid.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A composition comprising a hydrogel, the hydrogel comprising a polymer of formula (1): 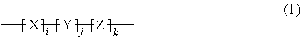
where
X comprises one or more repeating units of formula (2): 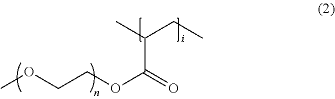
Y comprises one or more repeating units of formula (3): 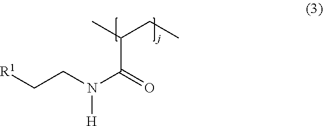
Z comprises one or more repeating units of formula (4): 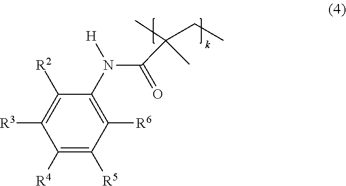
R¹ is H or OH;
R²-R⁶ each independently comprise H, C, or a heteroatom; and
n, i, j, and k, are each independently an integer from 1 to 4000;
wherein
at least one repeating unit of Z comprises BO₂H₂; and
the polymer of formula (1) comprises a number average molecular weight (M_{N}) obtained via gel permeation chromatography (GPC) from 70 kDa to 250 kDa.

2. The composition of claim 1, wherein R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl;
where R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.

3. The composition of claim 2, wherein the other functional groups, when present, are selected from the group consisting of amino, carboxyl, hydroxy, alkoxy, ketone, aldehyde, halogen, and a combination of two or more thereof.

4. The composition of claim 1, wherein R² of at least one repeating unit of Z is BO₂H₂.

5. The composition of claim 1, wherein R³ of at least one repeating unit of Z is BO₂H₂.

6. The composition of claim 1, wherein R⁴ of at least one repeating unit of Z is BO₂H₂.

7. The composition of claim 1, wherein R⁵ of at least one repeating unit of Z is BO₂H₂.

8. The composition of claim 1, wherein R⁶ of at least one repeating unit of Z is BO₂H₂.

9. The composition of claim 7, wherein R², R³, R⁴, and R⁶ of at least one repeating unit of Z are each H, and R⁵ of the at least one repeating of Z is BO₂H₂.

10. The composition of claim 1, further comprising at least one pluripotent stem cell.

11. The composition of claim 1, wherein the polymer of formula (1) comprises a number average molecular weight (M_{N}) obtained via gel permeation chromatography (GPC) from 100 kDa to 250 kDa.

12. The composition of claim 1, wherein a concentration of BO₂H₂ groups present on the polymer of formula (1) is from 0.25 mmol/g to 1 mmol/g.

13. The composition of claim 1, further comprising a diol-containing molecule.

14. The composition of claim 13, wherein the diol-containing molecule comprises a polymer comprising at least one set of vicinal hydroxyl substituents.

15. The composition of claim 14, wherein the polymer comprising at least one set of vicinal hydroxyl substituents comprises polyvinyl alcohol.

16. A method for culturing at least one cell on a hydrogel, the method comprising:
forming the composition of claim 1;
adding a diol-containing molecule to the composition;
adding the cell to the composition; and
allowing the BO₂H₂ to react with the diol-containing molecule, thereby forming the hydrogel.

17. The method of claim 16, wherein the cell is a stem cell.

18. The method of claim 17, wherein the cell is a pluripotent stem cell.

19. The method of claim 18, wherein the pluripotent stem cell is from a mammal.

20. The method of claim 19, wherein the mammal is a human.

21. The method of claim 16, wherein the diol-containing molecule comprises polyvinyl alcohol.
